(19) United States
(12) Patent Application Publication
(10) Pub. No.: US 2024/0349001 A1
(43) Pub. Date: Oct. 17, 2024

(54) METHOD AND SYSTEM FOR DETERMINING INDIVIDUALIZED HEAD RELATED TRANSFER FUNCTIONS

(71) Applicant: MCMASTER UNIVERSITY, Hamilton (CA)

(72) Inventors: Navid H. ZANDI, Hamilton (CA); Awny M. EL-MOHANDES, Hamilton (CA); Rong ZHENG, Hamilton (CA)

(21) Appl. No.: 18/580,344

(22) PCT Filed: Jul. 18, 2022
(86) PCT No.: PCT/CA2022/051112

§ 371 Date: Jan. 18, 2024

Related U.S. Application Data
(60) Provisional application No. 63/223,169, filed on Jul. 19, 2021.

(51) Int. Cl. H04S 7/00; G06N 3/0455

(52) U.S. Cl. CPC H04S 7/303; G06N 3/0455; H04S 2400/11; H04S 2400/15; H04S 2420/01; H04S 2420/03

(57) ABSTRACT

There is provided a system and method for determining individualized head related transfer functions (HRTF) for a user. The method including: receiving measurement data from the user, the measurement data generated by repeatedly emitting an audible reference sound at positions in space around the user and, during each emission, recording sounds received near each ear of the user, the measurement data including, for each emission, the recorded sounds and positional information of the emission; determining the individualized HRTF by updating a decoder of a trained generative artificial neural network model, the decoder receives the measurement data as input, the trained generative artificial neural network model including an encoder and the decoder, the generative artificial neural network model is trained using data gathered from a plurality of test subjects with known spectral representations and directions for associated HRTFs at different positions in space; and outputting the individualized HRTF.

### TECHNICAL FIELD

The following relates generally to auditory devices, and more specifically, to a method and system for determining individualized head related transfer functions.

### BACKGROUND

Head-Related Transfer Functions (HRTFs) represent an acoustic filtering response of humans' outer ear, head, and torso. This natural filtering system plays a key role in human binaural auditory systems that allows people to not only to hear but also to perceive the direction of incoming sounds. The HRTF characterizes how a human ear receives sounds from a point in space, and depends on, for example, the shapes of a person's head, pinna, and torso. Accurate estimations of HRTFs for human subjects are crucial in augmented or virtual realities applications, among other applications. Unfortunately, approaches for HRTF estimation generally rely on specialized devices or lengthy measurement processes. Additionally, using another person's HRTF, or a generic HRTF, will lead to errors in acoustic localization and unpleasant experiences.

### SUMMARY

In an aspect, there is provided a computer-executable method for determining an individualized head related transfer functions (HRTF) for a user, the method comprising: receiving measurement data from the user, the measurement data generated by repeatedly emitting an audible reference sound at positions in space around the user and, during each emission, recording sounds received near each ear of the user, the measurement data comprising, for each emission, the recorded sounds and positional information of the emission; determining the individualized HRTF by updating a decoder of a trained generative artificial neural network model, the decoder receives the measurement data as input, the trained generative artificial neural network model comprising an encoder and the decoder, the generative artificial neural network model is trained using data gathered from a plurality of test subjects with known spectral representations and directions for associated HRTFs at different positions in space; and outputting the individualized HRTF.

In a particular case of the method, the positions in space around the user comprise a plurality of fixed positions.

In another case of the method, the positions in space around the user comprise positions that are moving in space.

In yet another case of the method, the audible reference sound comprises an exponential chirp.

In yet another case of the method, the generative artificial neural network model comprises a conditional variational autoencoder.

In yet another case of the method, training of the conditional variational autoencoder comprises using the data gathered from the plurality of test subjects to learn a latent space representation for HRTFs at different positions in space.

In yet another case of the method, the decoder reconstructs an HRTF for the user's left ear and an HRTF for the user's right ear at a given direction from the latent space representation.

In yet another case of the method, a sparsity mask is input to the decoder to indicate a presence or an absence of parts of temporal data of the reference sound in a given direction.

In yet another case of the method, the individualized HRTF comprises magnitude and phase spectra.

In yet another case of the method, the phase spectra is determined by the generative artificial neural network model by learning real and imaginary parts of a Fourier transform of the HRTFs separately.

In yet another case of the method, an impulse response for the individualized HRTF is determined by applying an inverse Fourier transform on a combination of the magnitude and phase spectra.

In another aspect, there is provided a system for determining an individualized head related transfer functions (HRTF) for a user, the system comprising a processing unit and data storage, the data storage comprising instructions for the one or more processors to execute: a measurement module to receive measurement data from the user, the measurement data generated by repeatedly emitting an audible reference sound by a sound source at positions in space around the user and, during each emission, recording sounds received near each ear of the user by a sound recording device, the measurement data comprising, for each emission, the recorded sounds and positional information of the sound source; a machine learning module to determine the individualized HRTF by updating a decoder of a trained generative artificial neural network model, the decoder receives the measurement data as input, the trained generative artificial neural network model comprising an encoder and the decoder, the generative artificial neural network model is trained using data gathered from a plurality of test subjects with known spectral representations and directions for associated HRTFs at different positions in space; and an output module to output the individualized HRTF.

In a particular case of the system, the positions in space around the user comprise a plurality of fixed positions.

In another case of the system, the positions in space around the user comprise positions that are moving in space.

In yet another case of the system, the sound source is a mobile phone and the sound recording device comprises in-ear microphones.

In yet another case of the system, the generative artificial neural network model comprises a conditional variational autoencoder.

In yet another case of the system, training of the conditional variational autoencoder comprises using the data gathered from the plurality of test subjects to learn a latent space representation for HRTFs at different positions in space.

In yet another case of the system, the decoder reconstructs an HRTF for the user's left ear and an HRTF for the user's right ear at a given direction from the latent space representation.

In yet another case of the system, a sparsity mask is input to the decoder to indicate a presence or an absence of parts of temporal data of the reference sound in a given direction.

In yet another case of the system, the individualized HRTF comprises magnitude and phase spectra.

In yet another case of the system, the phase spectra is determined by the generative artificial neural network model by learning real and imaginary parts of a Fourier transform of the HRTFs separately.

In yet another case of the system, an impulse response for the individualized HRTF is determined by applying an inverse Fourier transform on a combination of the magnitude and phase spectra.

These and other embodiments are contemplated and described herein. It will be appreciated that the foregoing summary sets out representative aspects of systems and methods to assist skilled readers in understanding the following detailed description.

### BRIEF DESCRIPTION OF THE DRAWINGS

The features of the invention will become more apparent in the following detailed description in which reference is made to the appended drawings wherein:

FIG. 1 is a schematic diagram of a system for determining individualized head related transfer functions, in accordance with an embodiment;

FIG. 2 is a flow chart of a method for determining individualized head related transfer functions, in accordance with an embodiment;

FIGS. 3A to 3C show example HRTFs in time and frequency domains;

FIG. 4 illustrates an example pictorial overview of the method of FIG. 2;

FIG. 5 is a diagram illustrating inputs and outputs of a conditional variational autoencoder (CVAE) model;

FIG. 6A is a diagram showing an encoder of the CVAE of FIG. 5;

FIG. 6B is a diagram showing a decoder of the CVAE of FIG. 5;

FIG. 7A is a diagram illustrating 26 basis vectors spread evenly around a sphere, where for each desired direction, four surrounding points are identified and the desired direction is represented as a weighted average of its four neighboring basis vectors;

FIG. 7B is a diagram illustrating one-hot vector encoding of the subjects, where the last element is set to zero during training, and is 1 during individualization;

FIG. 8 illustrates a diagram of individualization of the decoder with a new user's data;

FIG. 9 is a diagram illustrating notations used in determining sound direction;

FIG. 10A is a diagram illustrating an example of a location of the reference angle, in the horizontal plane, at ITD=0;

FIG. 10B is a diagram illustrating an example of geometric techniques that can be used to determine lₛₕ|lₛ;

FIG. 10C is a diagram illustrating an example of a location of a reference vertical angle at ITDₘₐₓ;

FIGS. 11A to 11D illustrate example charts of comparisons of ground truth HRTFs and HRTFs with and without individualization for a subject at four different locations;

FIGS. 12A to 12C illustrate charts showing LSD errors for different subjects and with different measurement locations;

FIGS. 13A to 13D illustrate charts showing ground truth HRTFs and HRTFs with and without individualization using only HRTFs from locations in the user's frontal semisphere;

FIG. 14 is a diagram illustrating an example of ground truth for directions;

FIGS. 15A and 15B illustrate charts showing median, 25th, and 75th percentiles of azimuth and elevation angles estimations, respectively;

FIGS. 16A to 16D illustrate charts showing results of individualization using measurements data from one subject for different azimuths and elevations;

FIG. 17 is a diagram illustrating 12 azimuth and 2 elevations located around the user;

FIG. 18A is a diagram showing that for a continuous movement of a sound source, an arc is generated that is covered by the sound source during the playback;

FIG. 18B is a diagram showing, for a continuous movement of a sound source, sparsity in components of the received signal;

FIG. 19A is a diagram showing an example of an encoder;

FIG. 19B is a diagram showing an example of a decoder; and

FIG. 20 shows an illustrative example of an approach to HRTF individualization.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following relates generally to auditory devices, and more specifically, to a method and system for determining individualized head related transfer functions.

Embodiments of the present disclosure advantageously provide an approach for head related transfer function (HRTF) individualization. Advantageously, embodiments of the present disclosure can be implemented using commercial (non-specialized) off-the-shelf personal audio devices; such as those used by average users in home settings. The present approaches provide a generative neural network model that can be individualized to predict HRTFs of new subjects, and a lightweight measurement approach to collect HRTF data from sparse locations relative to other HRTF approaches (for example, on the order of tens of measurement locations).

Embodiments of the present disclosure provide an approach for HRTF individualization that makes it possible for individuals to determine an individualized HRTF at home, without specialized/expensive equipment. The present embodiments are substantially faster and easier than other approaches, and able to be conducted using commercial-off-the-shelf (COTS) devices. In some embodiments, a conditional variational autoencoder (CVAE), or other types of generative neural network models, can be used to learn a latent space representation of input data. Given measurement data from relatively sparse positions, the model can be adapted to generate individualized HRTFs for all directions. The CVAE model of the present embodiments has a small size, making it attractive for implementation on, for example, embedded devices. After training the model, for example using a public HRTF dataset, the HRTFs can be accurately estimated using measurements from, for example, as low as 60 locations from the new user. In a particular embodiment, two microphones 130 are used to record sounds emitted from a mobile phone. Positions of the phone can be estimated from on-board inertial measurement units (IMUs) in a global coordinate frame. To transform the position into a subject-specific frame, the interaural time difference (ITD) of the sound emitted by the mobile phone at the in-ear microphones 130 and geometric relationships among the subject's head, shoulder and arms are utilized. No anthropometric information is required from users. The total measurement can be completed in, for example, less than 5 minutes; which is substantially less than other approaches.

Humans' binaural system endows people the ability not only to hear but also to perceive the direction of incoming sounds. Even in a cluttered environment, like in a restaurant or a stadium, humans are capable of separating and attending to individual sound sources selectively. Different cues are used to determine the location of sound sources. Interaural cues like ITD and interaural level difference (ILD), both direction dependent, represent the time and intensity differences between the sounds received by the left and right ears of a subject, respectively. The ITD is zero when the distances that a sound travels to the ears are equal (directly in front of the head, or in the back), but increases as the sound moves toward one of the sides. The maximum ITD that one can experience depends on the size of one's head. The same is true for ILD: as a sound goes toward the sides, the level difference at one's two ears becomes higher. Spectral cues depend on the direction of the incoming signal as well as human physical features, such as the shapes and sizes of one's pinna, head, and torso.

Humans' ability to localize sound is attributed to the filtering effects of human ear, head and torso, which are direction and frequency dependent, and are described by head related transfer function (HRTF). HRTF characterizes the way sounds from different points in space are perceived by the ears, or in other words, a transfer function of the channel between a sound source and the ears. HRTF is typically represented in the frequency domain, and its counterpart in the time domain is called head related impulse response (HRIR). Consequently, HRTF is a function of the angles of an incoming sound (usually azimuth and elevation angles are used to define the location in three-dimensional (3D) interaural coordinates), and frequency, and is defined separately for each ear.

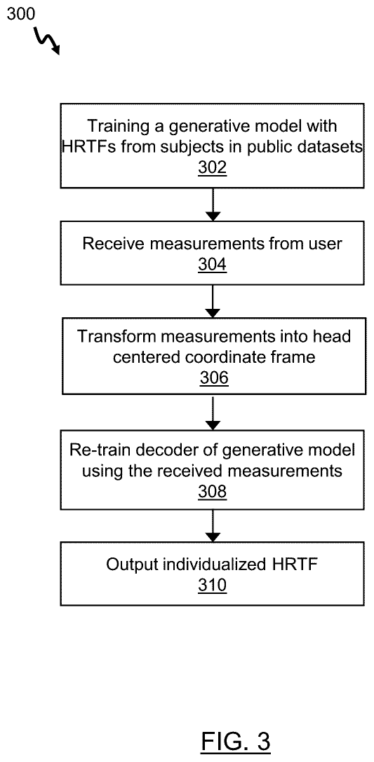

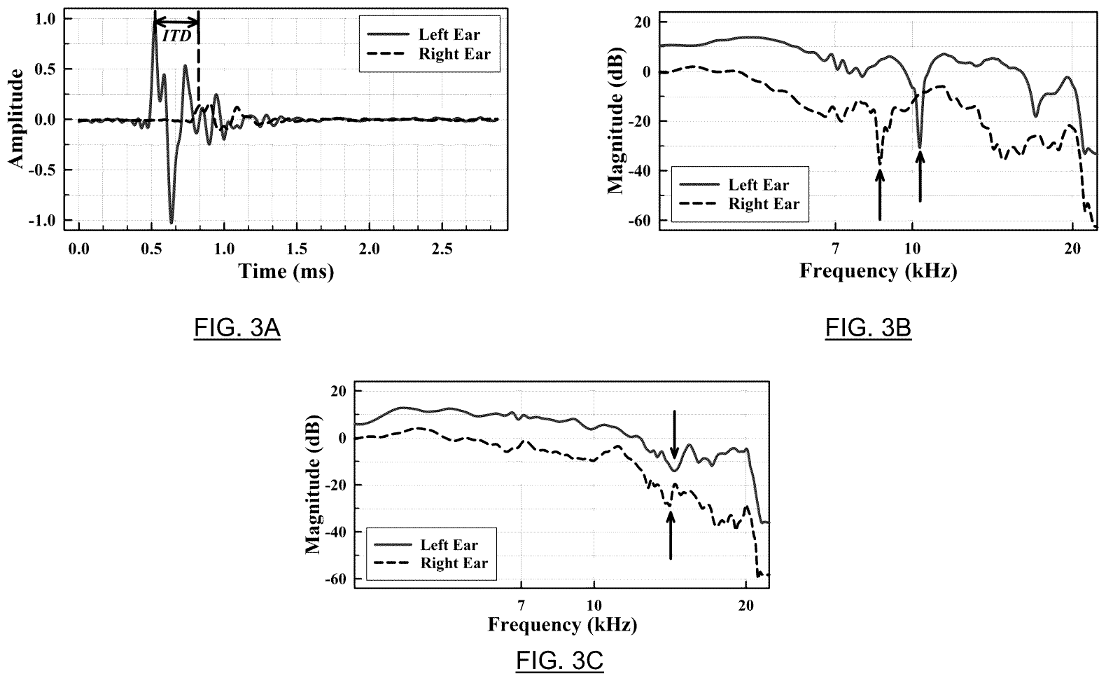

An example of HRIR and HRTF is illustrated in FIGS. 3A to 3C, for left and right ears. FIG. 3A shows HRIR at azimuth=45° and elevation=−5.76° (the time difference between the onsets of the signals at the two ears is the ITD). FIG. 3B shows HRTF at azimuth=45° and elevation=−5.7°. FIG. 3C shows HRTF at azimuth=45° and elevation=54.72° (some notches are marked with arrows). The notches appear at higher frequencies as the position of the sound moves toward the top of one's head.

As shown in FIG. 3B, many peaks and notches can be observed. As the position of the sound source goes toward the top of the head, the frequencies of spectral notches become higher (as illustrated in FIG. 3C). These notches are deeper near the horizontal plane (where the elevation angle is zero), and shallower above the plane. The perception of the elevation angle of a sound is related to the spectral notches and peaks above 5 KHz. On the other hand, ITD and ILD are the two main cues for 82 lateral localization, and they are directly affected by human's HRTF.

Emerging technologies such as Augmented Reality (AR), Virtual Reality (VR) and Mixed Reality (MR) systems use spatialization of sounds in three-dimensions (3D), to create a sense of immersion. To reproduce the effects of a sound from a desired incoming position, the sound waveform (e.g., a mono sound) is filtered by the left and right HRTFs of a target subject of this position, and played through a stereo headphone (or a transaural system with two loud speakers). Consequently, the sound scene (or the location that the sound comes from as perceived by the listener) can be controlled, and a sense of immersion is generated. Another important application that benefits from the knowledge of HRTFs is binaural sound source localization; which mainly can be used in robotics or used in earbuds as an alert system for users. However, since HRTFs are highly specific to each person, using another person's HRTFs, or a generic HRTF, can lead to localization errors and unpleasant experiences for humans. However, since HRTFs depend on the location of the sound, direct measurements are time-consuming and generally require special equipment. A substantial advantage of the present embodiments is providing an efficient mechanism to estimate subject-specific HRTFs, also referred to HRTF individualization.

Using generic HRTFs can be a substantial source of errors in many applications that use HRTFs. Approaches to individualize HRTFs can be grouped into four main categories:

(1) Direct Methods. The most obvious solution to obtaining individualized HRTFs for a subject is to conduct dense acoustic measurements in an anechoic chamber. One or several loud speakers are positioned at each direction of interest around the subject with microphones placed at the entrance of ear canals to record the corresponding impulse response. The number of required speakers can be reduced by installing them at different elevations on an arc, and rotating the arc to measure at different azimuths. This approach requires special devices and setups. The measurement procedure can be overwhelming to test subjects (often having to sit still for a long time). To accelerate the process, the Multiple Exponential Sweep Method (MESM) can be employed, where reference signals are overlapped in time. However, this method requires a careful selection of timing to prevent superposition of different impulse responses. An alternative way is the so-called reciprocal method, in which two small speakers are placed inside the subject's ears, and microphones are installed on an arc. This accelerates the measurement time, but has its own limitations, such as the speakers in the ears cannot produce too loud sounds as it may damage the person's ears (low SNR on the final measurements). The use of continuous measurements can also be performed using measurement in an anechoic room, such that at a rotation speed of 3.8°/s, no audible differences are experienced by subjects compared to step-wise measurement. In other cases, instead of moving her whole body, a subject is asked to move her head in different directions, with the head movements tracked by a motion tracker system. Long measurement time often leads to motion artifacts due to subject movements during the measurements.

(2) Simulation-based Methods. A second category of HRTF individualization can utilize numerical simulations of acoustic propagation around target subjects. To do so, a 3D geometric model of a listener's ears, head, and torso is needed, either gathered through 3D scans or 3D reconstruction from 2D images. Approaches, such as, finite difference time domain, boundary element, finite element, differential pressure synthesis, and raytracing are employed in numerical simulations of HRTFs. The accuracy of the 3D geometric model as inputs to these simulations is key to the accuracy of the resulting HRTFs. In particular, ears should be modeled more accurately than the rest of the body. Objective studies have reported good agreement between the computed HRTFs in simulation-based methods, and those from fine-grained acoustic measurements. Numerical simulations tend to be compute intensive. Most approaches require special equipment such as MRI or CT for 3D scan, and are thus not accessible to general commercial users. 3D reconstruction from 2D images eliminates the need for specialized equipment but at the expense of lower accuracy.

(3) Indirect Methods Using Anthropometric Measurements. HRTFs generally rely on the morphology of the listener. Therefore, many approaches try to indirectly estimate HRTFs from anthropometric measurements. Methods in this category tend to suffer the same problem as simulation-based methods in their need for accurate anthropometric measurements, which are often difficult to obtain. Some methods can be further classified into three subcategories:
(a) Adaptation: Starting from a non-individualized HRTF, scaling in the frequency domain can be applied for individualization, where the scaling factors can be estimated from head and pinna measurements. Subjective evaluations on 9 to 11 subjects have been shown to have improved localization performance over non-individualized HRTFs. Further improvement can be achieved by combining frequency scaling with rotation in space to compensate for head tilt.
(b) Nearest neighbor selection: In these approaches, the nearest HRTF set in a dataset is first selected based on the anthropometric measurements. The distances between two subjects can be computed either directly from morphological parameters, or features output from a neural network.
(c) Regression: In these approaches, the objective is to establish a functional or stochastic relation between anthropometric parameters and characteristics parameters of HRTFs. Principle component analysis (PCA) is often used to reduce the dimensionality of input and/or output parameters. In some cases, a linear model is assumed and estimated. The HRTFs for a new subject are then predicted using the subject's anthropometric parameters through the model.

(4) Indirect Methods based on Perceptual Feedback. Beside using anthropometric parameters to identify closely matched subjects in a dataset, a fourth category of approaches utilizes perceptual feedback from target listeners. A reference sound which contains all the frequency ranges (Gaussian noise, or parts of a music) is convoluted with selected HRTFs in a dataset and played through a headphone to create 3D audio effects. The listener then rates, among these playbacks, how close the perceived location of the sound is to the ground truth locations. Once the closest K-subjects in the dataset are found, the final HRTF of the listener can be determined through: (a) selection, namely, to use the closest non-individualized HRTF from the dataset; or (b) adaptation, using frequency scaling with a scaling factor tuned by the listener's perceptual feedback and statistical methods with the goal of reducing the number of tuning parameters using PCA or variational autoencoders. Methods using perceptual feedback are particularly relevant to sound spatialization tasks in AR/VR. However, these methods generally suffer from long calibration time and imperfection of human hearing (e.g., low resolutions in elevation angles, difficulty to discriminate sounds in front or behind one's body).

Advantageously, embodiments of the present disclosure use a combination of direct and indirect approaches. Such embodiments use HRTF estimations at relatively sparse locations from a target subject (direct measurements) and estimates the full HRTFs with the help of a latent representation of HRTFs (indirect adaptation).

Several datasets are available for HRTF measurements using anechoic chambers. They differ in the number of subjects in the dataset, the spatial resolution of measurements, and sampling rates. A dataset from the University of California Davis CIPIC Interface Laboratory contains data from 45 subjects. With a spacing of 5.625°×5°, measurements were taken at 1250 positions for each subject. A set of 27 anthropometric measurements of head, torso and pinna are included for 43 of the subjects. A LISTEN dataset measured 51 subjects, with 187 positions recorded at a resolution of 15°×15°. The anthropometric measurements of the subjects, similar to the CIPIC dataset are also included. A larger dataset, RIEC, contains HRTFs of 105 subjects with a spatial of resolution 5°×10°, totaling 865 positions. A 3D model of head and shoulders is provided for 37 subjects. ARI is a large HRTF dataset with over 120 subjects. It has a resolution of 5°×5°, with 2.5° horizontal steps in the frontal space. For 50 of the 241 subjects, a total of 54 anthropometric measurements are available, out of which 27 measures are the same as those in the CIPIC dataset. An ITA dataset has a high resolution of 5°×5°, with a total of 2304 HRTFs measured for 48 subjects. Using Magnetic Resonance Imaging (MRI), detailed pinna models of all the subjects are available.

In the aforementioned datasets, with the exception of LISTEN, measurements were done using multiple speakers mounted on an arc. In LISTEN, measurements were done using only one speaker, that moves in the vertical direction. Measurements from different azimuth angles are done by having subjects turn their bodies around.

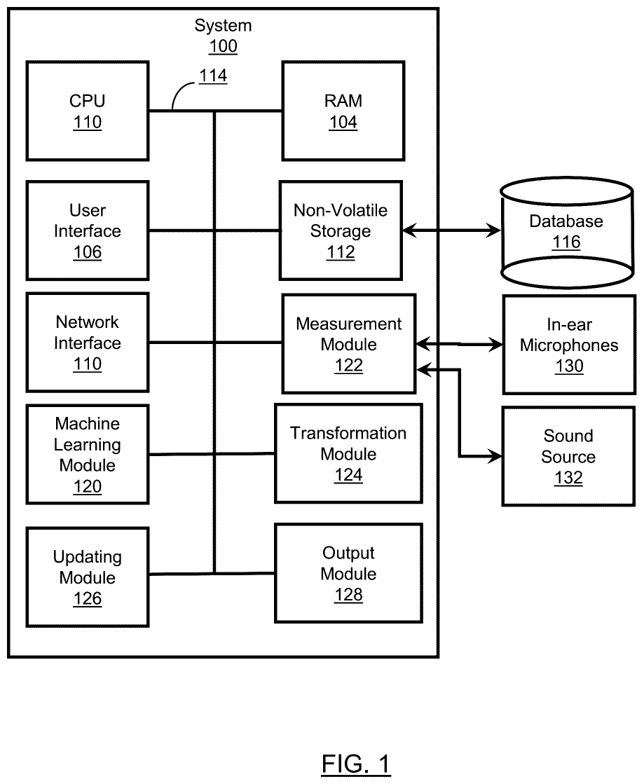

Referring now to FIG. 1, a system 100 for determining individualized head related transfer functions (HRTFs), in accordance with an embodiment, is shown. In this embodiment, the system 100 is run on a local computing device. In some cases, the local computing device can access content located on a server over a network, such as the internet. In further embodiments, the system 100 can be run on any suitable computing device; for example, a server. In some embodiments, the components of the system 100 are stored by and executed on a single computer system. In other embodiments, the components of the system 100 are distributed among two or more computer systems that may be locally or remotely distributed.

FIG. 1 shows various physical and logical components of an embodiment of the system 100. As shown, the system 100 can include a number of physical and logical components, including a central processing unit (“CPU”) 102 (comprising one or more processors), random access memory (“RAM”) 104, a user interface 106, a network interface 110, non-volatile storage 112, and a local bus 114 enabling CPU 102 to communicate with the other components. CPU executes software, and/or an operating system, with various functional modules, as described below in greater detail. While the present embodiments describe a CPU 102, it is contemplated that the presently described functions can be executed via an embedded hardware implementation. RAM 104 provides relatively responsive volatile storage to CPU 102. The user interface 106 enables an administrator or user to provide input via an input device, for example a touch screen. The user interface 106 can also output information to output devices to the user, such as a display and/or speakers. The network interface 110 permits communication with other systems, such as other computing devices and servers remotely located from the system 100, such as for a typical cloud-based access model. Non-volatile storage 112 stores the operating system and programs, including computer-executable instructions for implementing the operating system and modules, as well as any data used by these services. Additional stored data, as described below, can be stored in a database 116. During operation of the system 100, the operating system, the modules, and the related data may be retrieved from the non-volatile storage 112 and placed in RAM 104 to facilitate execution.

In an embodiment, the system 100 includes a number of functional modules, each executed on the one or more processors 110, including a machine learning module 120, a measurement module 122, a transformation module 124, an updating module 126, and an output module 128. In some cases, the functions and/or operations of the machine learning module 120, the measurement module 122, the transformation module 124, the updating module 126, and the output module 128 can be combined or executed on other modules.

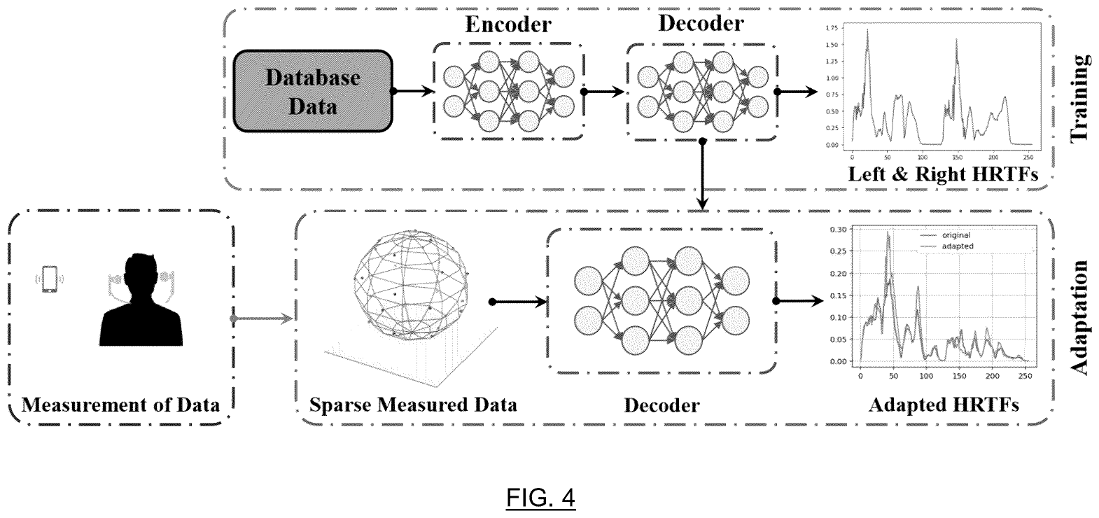

FIG. 3 illustrates a method 300 for determining individualized head related transfer functions, in accordance with an embodiment. FIG. 4 illustrates an example pictorial overview of the method 300. The method 300 generally includes collecting relatively sparse measurements from a target subject from a device and using a trained a CVAE (trained using HTRF data from existing public or private datasets) to determine an individualized HRTF for the user based on the relatively sparse measurements.

The approach of the system 100 to HRTF individualization adapts a generative neural network model trained from HRTFs from existing datasets using relatively sparse direct acoustic measurements from a new user. In a particular case, the machine learning module 120 uses a conditional variational autoencoder (CVAE); a type of conditional generative neural network model that is an extension of a variational autoencoder (VAE). However, in further cases, other suitable generative neural network machine learning models can be used. The CVAE has two main parts: (1) an encoder that encodes an input x as a distribution over a latent space p(z|x), and (2) a decoder that learns the mapping from the latent variable space to a desired output. To infer p(z) using p(z|x), which is not known, variational inference can be used to formulate and solve for an optimization problem. In some cases, for the ease of computation, p(z|x) can be modeled as a Gaussian distribution. In most cases, parameters estimation can be done using stochastic gradient variational Bayes (SGVB), where the objective function of the optimization problem is the variational lower bound log-likelihood; or any other suitable approach.

At block 302, the machine learning module 120 trains a CVAE network using data from a number of test subjects (e.g., from 48 test subjects in the ITA HRTF dataset), to learn a latent space representation for HRTFs at different positions (i.e., azimuth and elevation angles) in space. The CVAE network takes as inputs HRTFs from the left and right ears, the direction of the HRTFs, and a one-hot encoded subject vector. After training, the machine learning module 120 can use the decoder in the CVAE model to generate HRTFs for any subject in the dataset at arbitrary directions by specifying the subject index and direction vectors as inputs. However, it cannot generally be used to generate HRTFs for a specific user not part of the training dataset. To obtain individualized HRTFs, as described herein, the collected measurement data from the user is used.

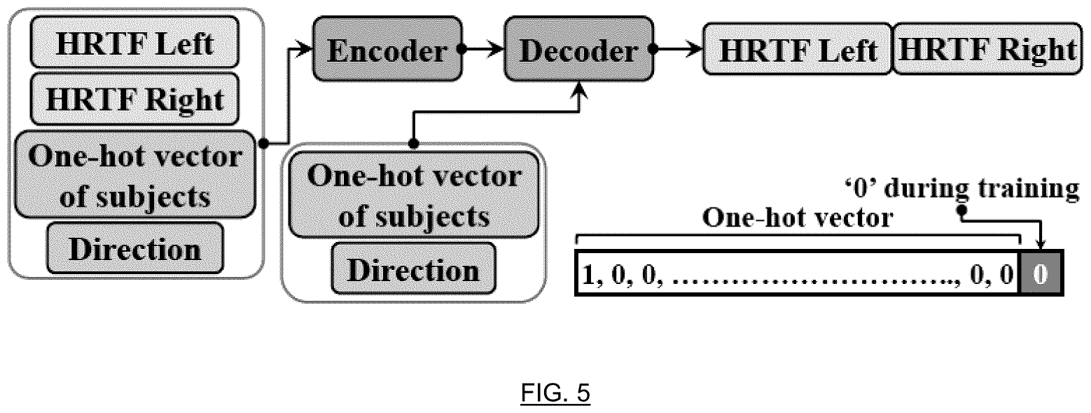

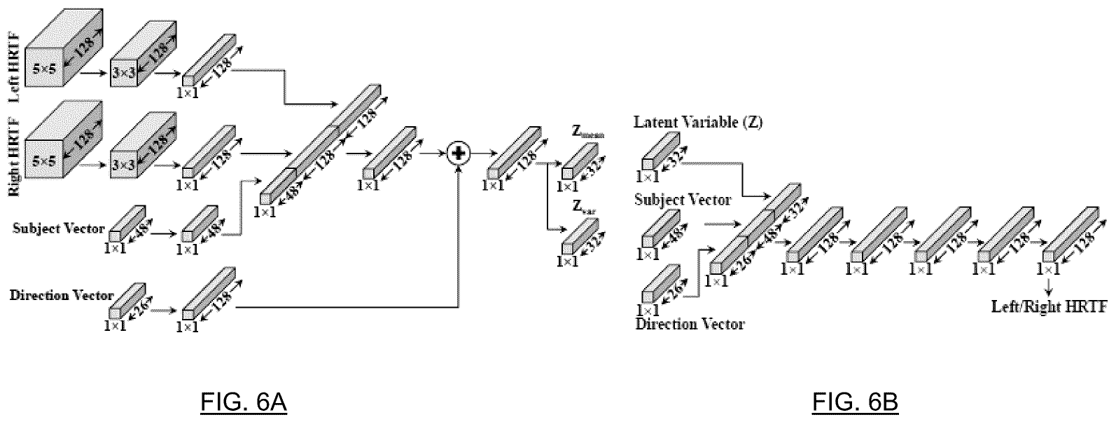

FIG. 5 illustrates an example diagram for the training and adaptation of the CVAE model for the present embodiments. The CVAE model consists of an encoder network and a decoder network. FIGS. 6A and 6B illustrate a diagram of an architecture of the CVAE model, where FIG. 6A shows the encoder that encodes an input HRTF into a latent space representation, and FIG. 6B shows the decoder that reconstructs the input HRTF based on its direction and subject vector.

The encoder can be used to extract a relation between HRTFs of neighboring angles in space, while learning the relationship between HRTF's adjacent frequency and time components at the same time. In some cases, this is achieved by constructing two 5×5 grids of HRTFs for left and right ears from neighboring angles as the input, centered at a desired direction D. Each of the left and right ear HRTFs grids can go through two layers of 3D convolution layers to form the HRTF's features, which helps to learn the spatial and temporal information.

Other inputs to the encoder can include a vector (e.g., of size 26) for the desired direction D, and a subject ID that can be a one-hot vector encoding of the desired subject among all available subjects in a training dataset; for whom the system constructs the HRTF grids. Length of the one-hot vector is N+1, N being the number of subjects available in the training dataset. The one extra element is reserved for the new unseen subject that is not in the dataset, whose individualized HRTFs the system will predict using the machine learning model. The direction vector can be constructed by mapping the data from azimuth and elevation angles in spherical coordinates by defining evenly dispersed basis points on the sphere (e.g., 26 points), and representing each desired direction with a weighted average of its four enclosing basis points. In the direction vector (D), the corresponding values for the surrounding basis points equals to the calculated weights, while the other values are set to zero. The output of encoder is a 1-D latent vector (z), for example, of size 32.

The decoder can reconstruct left and right ear HRTFs at the desired direction D from the latent space. Latent space vector, direction vector and subject vector are concatenated to form the input of the decoder. By including a sparsity mask as an extra condition to the network at later layers, in some cases, the decoder is able to learn temporal data sparsity. Sparsity mask is either “o” or “1”; indicating presence or absence of the parts of temporal data (frequency components) of the reference sound in the corresponding direction; which is expected when the sound source moves during HRTF measurements. This sparsity mask can also be used as part of the loss function. It forces the network to only update those weights of the model during backpropagation that correspond to temporal components of the HRTF that are present at the desired direction D (those with value of “1” in the sparsity mask).

The model predicts the magnitude and phase spectra of HRTFs at the output. The phase spectra is estimated by learning the real and imaginary parts of the Fourier transform of HRTFs separately. In example experiments conducted by the present inventors, it was found that applying a p-law algorithm to the magnitude spectra at the output layer leads to lower HRTF prediction error. The final impulse response can be reconstructed by applying the inverse Fourier transform on combination of magnitude and phase spectra.

The inherent high fluctuations of the audio signals makes their estimation hard with neural networks. The common activation functions used in neural networks, like ReLu or Elu, have difficulties following the temporal structure of audio signals. By using a periodic activation function, the model can better preserve this fine temporal structure.

For training, the encoder network takes three inputs: spectral representations of the HRTFs of a training subject, an associated direction vector, and a one-hot vector representing that training subject. For each training subject and each direction in the dataset, the machine learning module 120 applies a fast Fourier transform to the HRTFs from, for example, 5×5 grid points centred at the respective direction. The grid points are separated by, for example, ±0.08η in azimuth and elevation angles and are evenly spaced. The machine learning module 120 determines power spectrum density for the HRTF at each grid point over, for example, 128 frequency bins giving rise to, in this example, a 5×5×128 tensor for each of the left and right ears. The two tensors are separately passed through two convolutional neural network (CNN) layers to form HRTF features.

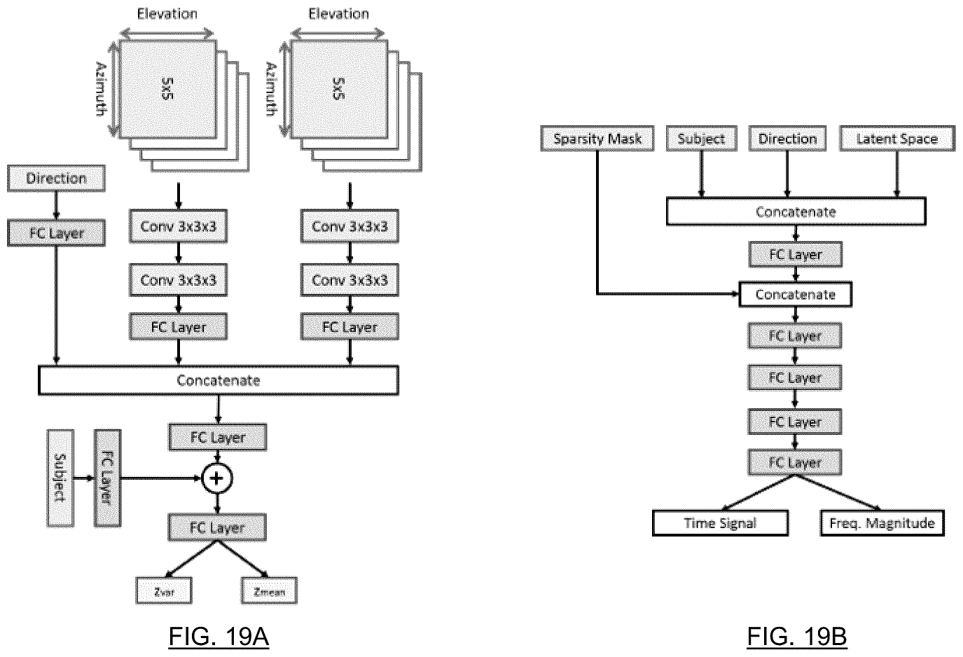

Advantageously, this approach to generating the HRTF substantially improves the time domain characteristics of HRTF; which leads to improved HRFT estimation accuracy and naturalness of sounds in spatial audio. FIGS. 19A and 19B illustrate HRTF model architecture for the machine learning model, in accordance with an embodiment. FIG. 19A shows an example of an encoder to compress data into a lower dimension latent space. FIG. 19B shows an example of a decoder to generate the HRTF at a desired direction conditioned on the subject vector, and the sparsity mask.

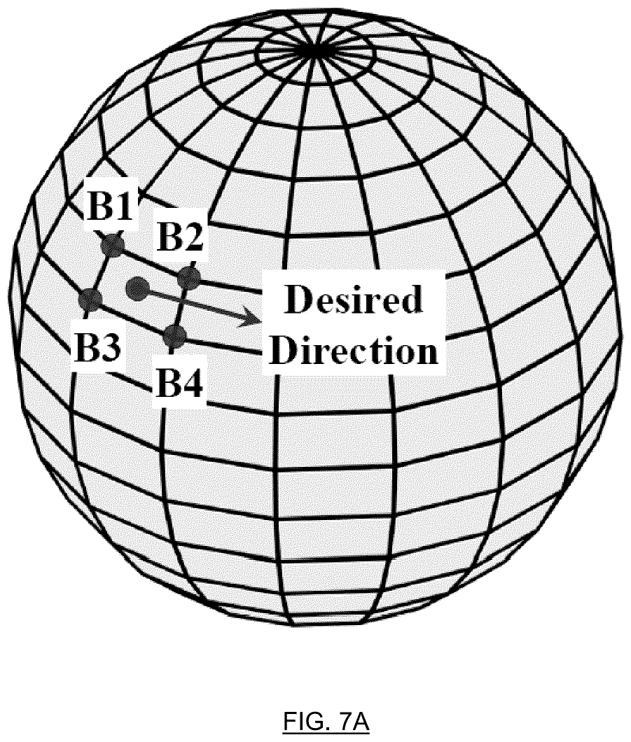

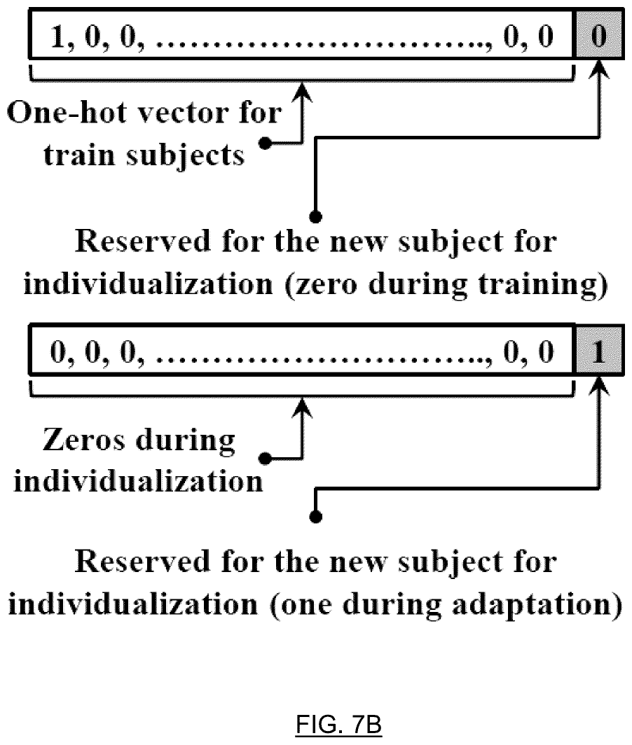

As illustrated in the example of FIG. 7B, the subject/user ID can be encoded as a one-hot vector; however, any suitable encoding can be used. Let N be the number of subjects in the training set. The vector is of length N+1. The i-th subject is thus associated with a vector with all elements but the i-th one being zero. The (N+1)th element in the vector is reserved for individualization. The last element is set to zero when training the CVAE. Each one-hot encoded subject vector goes through a fully-connected layer, and then is concatenated with the output of the CNN layers from the preceding step. The concatenated tensor then goes through another fully-connected layer. The next input to the encoder is a direction vector of the corresponding HRTF. In a particular case, instead of representing the direction in [symbol] ³, a vector in [symbol] ²⁶ is used, where the basis vectors correspond to 26 evenly distributed points on the sphere as shown in FIG. 7A. In the case of 26 evenly distributed points, they are distributed such that there is a point at each of the six azimuth angles for each of four elevation angles, and a point at the top and the bottom of the sphere. In further cases, any suitable number of distributed points can be used, with varying levels of added or reduced complexity.

In the case of 26 evenly distributed points, FIG. 7A illustrates that the 26 basis vectors are spread evenly around the sphere; where for each desired direction, the four surrounding points are identified, and the desired direction is represented as a weighted average of its four neighboring basis vectors. For each direction u, four enclosing neighbouring points (B1, B2, B3, B4) are identified, and the weights for the basis vectors (w1, w2, w3, w4) are determined as:

$$s = ( {ϕ}_{u} - {ϕ}_{B 2} ) / ( {ϕ}_{B 1} - {ϕ}_{B 2} ) \qquad ( 1 )$$

$$t = ( {θ}_{u} - {θ}_{B 3} ) / ( {θ}_{B 1} - {θ}_{B 3} )$$

$$( {w}_{1} , {w}_{2} , {w}_{3} , {w}_{4} ) = ( st , ( 1 - s ) t , s ( 1 - t ) , ( 1 - s ) ( 1 - t ) ) \text{?}$$

$$\text{?} \text{indicates text missing or illegible when filed}$$

where ϕ and θ are the azimuth and elevation angles of the corresponding points.

The weights for directions other than the four surrounding basis vectors are set to zero. As an example, consider a direction (azimuth, elevation)=(17.5°, 0°). Its enclosing basis vectors correspond to B1=(60°,18°), B2=(0°, 18°), B3=(60°, −18°), B4=(0°, −18°), in the spherical coordinate frame. The corresponding weights are given by:

w₁=0.35416667,w₂=05416667

w₃=0.14583333,w₄=0.4583333.

Compared to representations in [symbol] ³, the above described representation is more suitable [symbol] for processing by the present neural networks as they are sensitive to binary like activation. Each direction vector in [symbol] ²⁶ goes through a fully-connected layer, and is then summed with the output from the preceding step, as the encoder input, which is mapped into the latent variable space.

For training of the decoder, the machine learning module 120 concatenates an output from the encoder with training subject and direction features, and passes it through fully-connected layers (e.g., 5) of the same size, and an output layer, to generate HRTF sets of the left and right ears for each training subject in the desired direction.

In some cases, exponential-linear activation functions can be used after each layer in the encoder and the decoder, except for the final output layer that can use a sigmoid function. In further cases, other suitable activation and output functions can be used. The network architecture employed by the machine learning module 120 differs from a typical CVAE model in two or more important ways. Firstly, HRTF generation is performed as a regression problem. Thus, the outputs of the decoder are floating point vectors (e.g., of size 256, with 128 for each ear). Using such outputs of the decoder drastically decreases the number of parameters in the network due to the reduced number of units in the output layer. Secondly, no adaption layers need be included, which further reduces the number of learning parameters. As a result, in an example, the total number of parameters of the present CVAE model is 367,214; while other typical CVAE models can have, for example, 1,284,229,630 parameters. Advantageously, a lower number of training parameters generally implies shorter training time and higher data efficiency.

At block 304, the measurement module 122 receives measurement data from a user. Unlike other step-wise approaches, continuous HRTF measurement by the measurement module 122 does not require a specialized facility; such as anechoic rooms and stationary or moving loud speakers. Instead, for example, any device with speakers and inertial measurement unit (IMU) sensors can function as a sound source. For the purposes of this disclosure, reference will be made to a smartphone; however any suitable device can be used. Advantageously, the continuous measurement approach allows the total measurement time to be substantially reduced and reduces muscle fatigue of the user due to not have to keep the sound source still, as described herein.

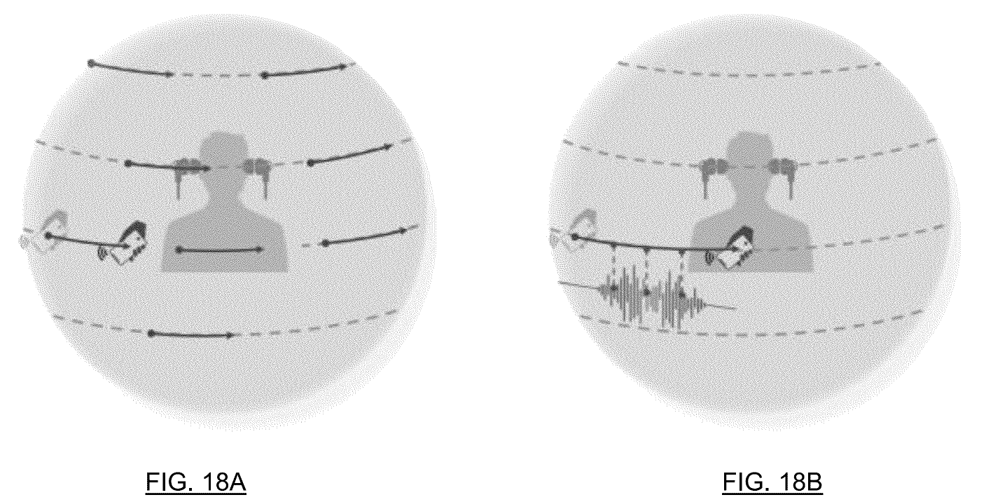

In an example of a continuous measurement approach, to perform the measurements, a user can hold a sound source 132 (such as a user's mobile phone) in hand and stretch out that arm as far as possible, while wearing two in-ear microphones 130 in their left and right ears. The user can continuously move the sound source 132 (such as a speaker on the user's mobile phone) around in arbitrary directions during periodic playbacks of a reference sound. In a particular case, an exponential chirp signal is played repetitively and is recorded each time by the two in-ear microphones 130. Since the phone moves along arcs centered at the user's should joint, the resulting trajectories lie on a sphere as illustrated in FIG. 18A. FIG. 18B illustrates sparsity in the components of the received signal. Each position in space corresponds to a specific component of the played signal. As described herein, a direction finding algorithm is used to determine the direction of the sound source 132 at points in time with respective to the user's head. This allows the system to tag segments of the recorded sound with the directions of the sound.

In the continuous measurement approach, partial portions of the exponential chirps are received at directions along the moving trajectory of the sound source. In order to determine directions, the system can discretize continuous time into slots, where each slot maps to a frequency range in the received chirp signal. As described herein, spatial masks of binary values can be used in the neural network model such that, for a specific direction, the system can define a mask to indicate which portion of the chirp signal is received; and null out the rest with zeros.

In the above example, the user wears in-ear microphones 130. The measurement module 122 instructs a reference signal to be emitted from a sound source 132 (such as a speaker on the user's mobile phone). Sounds impinging upon in-ear microphones 130 are recorded while the reference signal is being emitted and the recorded sounds are communicated to the measurement module 122. During reference signal emission and recording, the user, or another person, freely moves the sound source 132 (such as with the user's right and left hands) in space.

In a particular case, measurement requires two in-ear microphones 130, one for each ear, to record the sounds impinging on the user's ears, and requires the sound source 132 to play sounds on-demand. The sound source 132 includes sensors to estimate the location of the emitted sounds, such as an inertial measurement unit (IMU) on a mobile phone.

In an example of step-wise measurement, instead of continuous measurement, during measurements, the user needs to put the two in-ear microphones 130 in their ears, hold the sound source 132 in their hand, and stretch out their arm from their body. In some cases, where the sound source 132 is for example a mobile phone, it is beneficial to hold the long edge of the mobile phone parallel to the extension of the user's arm. During measurement, the user's torso remains approximately stationary while they move their upper limbs. As the user moves their arm around, the user can pause at arbitrary locations and where a pre-recorded sound is emitted using the sound source 132. In a particular case, the pre-recorded sound can be an exponential sine sweep signal; which allows better separation of nonlinear artifacts caused by acoustic transceivers from useful signals compared to white noise or linear sweep waves. Once the emitted pre-recorded sound finishes playing, the user can proceed to another location where the pre-recorded sound is emitted again. This movement and playing of the pre-recorded sound can be repeated multiple times. In general, no special motion pattern for the arm is required, however, it may be preferable if the user tries to cover as much range as possible while keeping their shoulder at approximately the same location. In some cases, the multiple movements and playing of the pre-recorded sound is repeated for both hands in order to have the maximum coverage.

During measurement, at each position of the playing of the pre-recorded sound, two sources of information are obtained by the measurement module 122: (1) the recorded sounds in the two microphones 130, and (2) the position in space that the reference sound is played by the sound source 132. Using these two pieces of information, the system 100 can determine the individualized HRTFs by deconvolving the reference sound from the recorded sounds in both ears. The directions of sound sources 132 can be determined without user anthropometric parameters and specialized equipment.

At each position of the playing of the pre-recorded sound, IMU sensor data is received and stored to determine the orientation of the sound source 132 in space. Any suitable sensor fusion technique can be utilized for this purpose; such as the Mahony filter and the Madgwick filter, both with the ability to mitigate magnetic interference from surrounding environments. However, the resulting orientation is with respect to a global coordinate frame (GCF). To determine the direction of the sound sources 132, at block 306, the transformation module 124 performs transformations to determine the sound source's azimuth and elevation angles in a head centered coordinate frame (HCF).

The key difference between step-wise and continuous measurements is that in the former, all frequency bins in the power spectrum of the reference sound can be emitted at approximately the same set of locations. In the latter, in contrast, different portions of the same sound can be played back at different locations. In other words, from each location along the trajectories, only a subset of the frequency bins can be recorded as illustrated in FIG. 18B. In this way, continuous measurements can accelerate the measurement procedure since users do not have to wait at each measurement location during playback. However, special care should be taken when training and individualizing HRTFs in the continuous approach.

For acoustic channel identification, different reference sounds can be used; for example, white noise and chirps. In a particular case, exponential chirps can be used due to its ability to separate electro-acoustic subsystem artefacts from the desired impulse responses. The artefacts arise from the non-linearity of the impulse response of speaker and microphone. An exponential chirp is given by:

f(t)=f₀k^{t}

where f₀ is the starting frequency, and k is the rate of exponential change in frequency. Let f₁ be the ending frequency and T be the chirp duration:

$$k = {( \frac{{f}_{1}}{{f}_{0}} )}^{\frac{1}{T}}$$

The chirp interval T has a direct impact on the data collection time and channel estimation. A small T leads to shorter data collection time. However, if the T is too small (and consequently the signal duration is short), the received signal-noise-ratio (SNR) is low. The reference signal is played repetitively, with short periods of silence in between each playback. These silence periods allow room reverberations to settle before the next reference signal is played.

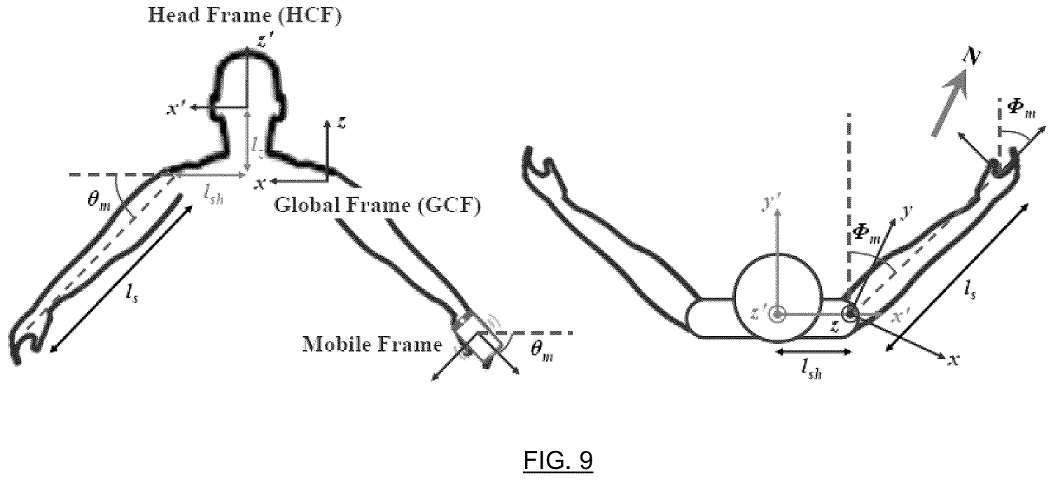

As illustrated in FIG. 9, notations are defined as followed for determining the HCF:
The HCF is a coordinate frame whose origin is at the centre of the head between a user's two ears. Its y- and x-axes are both in a horizontal plane pointing to the front and right sides of the user's body, respectively. The z-axis is vertical pointing upward.
The GCF is a coordinate frame centered on the shoulder joint of the sound source 132 holding hand with the y- and x-axes pointing to geographical North and East, respectively. Its z-axis is vertical pointing away from the center of the earth. By default, the GCF is centered on the right shoulder joint unless otherwise specified.
α is the rotation angle around the z-axis from GCF to HCF clockwise.
ϕₘ and θₘ are, respectively, the azimuth (with respect to the geographical North) and elevation angles of the sound source 132 in the GCF (such as the mobile phone's long edge as aligned with the user's arm).
ϕₘ′ and θₘ′ are, respectively, the azimuth and elevation angles of the sound source 132 in the HCF (such as the mobile phone's long edge).
lₛₕ is the shoulder length of the user from their left or right shoulder joint to the centre of their head.
lₛ is the distance from the user's left and right shoulder joint to sound source 132;
l_{z} is the vertical distance between the centre of user's shoulders and the centre of their head.

Consider a point P in space, whose coordinates in HCF and GCF are, respectively, (x′,y′,z′) and (x,y,z). From the above notation definitions, GCF and HCF can be related by translations on x- and y-axes by lsh and lz and a rotation around the z-axis clockwise of an angle α. Specifically:

$${[ {x}^{′} , {y}^{′} , {z}^{′} ]}^{T} = {{\underbrace{[ \begin{matrix}cos α & - sin α & 0 \\ sin α & cos α & 0 \\ 0 & 0 & 1\end{matrix} ]}}_{{R}_{z} ( α )} [ x , y , z ]}^{T} + {[ {l}_{sh} , 0 , - {l}_{z} ]}^{T} , \qquad ( 2 )$$

where R_{z}(α) is a rotation matrix around the z-axis.

When the sound source 132 is at azimuth m and elevation angle θₘ in the GCF, its Cartesian coordinates are (lₛ cos θₘ sin ϕₘ, lₛ cos θₘ cos ϕₘ, lₛ sin θₘ). From Equation (2), its Cartesian coordinates in the HCF are thus:

$$( {l}_{s} cos {θ}_{m} sin {ϕ}_{m} cos α - {l}_{s} cos {θ}_{m} cos {ϕ}_{m} sin α + {l}_{sh} , \text{?} cos {θ}_{m} sin {ϕ}_{m} sin α + \text{?} cos {θ}_{m} cos {ϕ}_{m} cos α , \text{?} sin {θ}_{m} - \text{?} )$$

$$\text{?} \text{indicates text missing or illegible when filed}$$

The azimuth and elevation angles of the sound source 132 in the HCF are given by:

$$( 3 )$$

$${ϕ}_{m}^{′} = arc tan ( \frac{\text{?} cos {θ}_{m} sin {ϕ}_{m} cos α - \text{?} cos {θ}_{m} cos {ϕ}_{m} sin α + {l}_{sh}}{\text{?} cos {θ}_{m} sin {ϕ}_{m} sin α + \text{?} cos {θ}_{m} cos {ϕ}_{m} cos α} ) = arc tan ( \frac{cos {θ}_{m} sin {ϕ}_{m} cos α - cos {θ}_{m} cos {ϕ}_{m} sin α + \frac{\text{?}}{\text{?}}}{cos {θ}_{m} sin {ϕ}_{m} sin α - cos {θ}_{m} cos {ϕ}_{m} cos α} )$$

$$and$$

$${θ}_{m}^{′} = arc tan ( \frac{( \sqrt{\begin{matrix}{( cos {θ}_{m} sin {ϕ}_{m} cos α - cos {θ}_{m} cos {ϕ}_{m} sin α + \frac{\text{?}}{\text{?}} )}^{2} + \\ {( cos {θ}_{m} sin {ϕ}_{m} sin α + cos {θ}_{m} cos {ϕ}_{m} cos α )}^{2}\end{matrix}}}{( sin {θ}_{m} - \frac{\text{?}}{\text{?}} )} ) \qquad ( 3 )$$

$$\text{?} \text{indicates text missing or illegible when filed}$$

To determine the estimated HRFT for the user, with appropriate location labels, the system 100 needs to determine a relative position of the sound source 132 in comparison to the user. This is non-trivial without the knowledge of anthropometric parameters of the user. Advantageously, the transformation module 124 uses a sensor fusion technique, using Equation (3) and Equation (4), to transform device poses from a device frame of the sound source 132 to a body frame of the user. Using Equation (3) and Equation (4), the unknown parameters are α, lₛₕ/lₛ, and l_{z}/lₛ. Note that there is generally no need to know the exact values of lₛₕ, lₛ and l_{z}; instead, the ratios are generally sufficient. Advantageously, the present inventors have determined that these parameters can be determined without knowledge of anthropometric parameters.

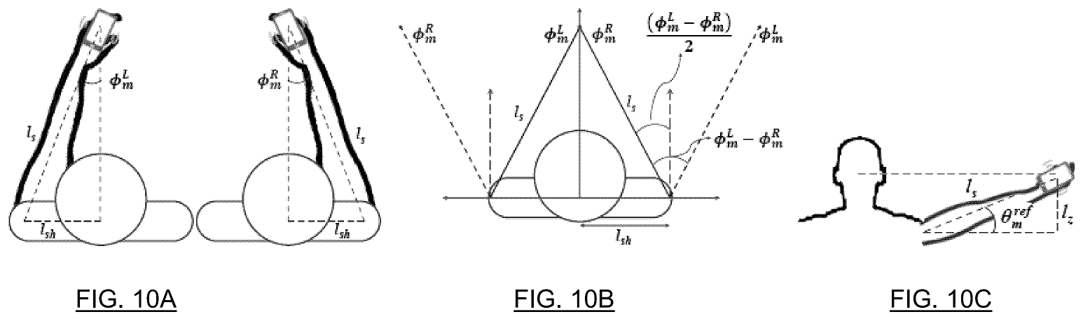

To estimate lₛₕ/lₛ, there are locations of the sound source 132 associated with a known azimuth or elevation angles in the GCF based on ITD measurements. FIGS. 10A to 10C illustrate an example of geometrical relations in the horizontal and frontal planes. Measurements are done using both right and left hands. A reference angle can be found when ITD=0 and |ITD| reaches its maximum. FIG. 10A illustrates an example of a location of the reference angle, in the horizontal plane, at ITD=0. FIG. 10B illustrates an example of geometric techniques that can be used to determine lₛₕ/lₛ. FIG. 10C illustrates an example of a location of a reference vertical angle at ITDₘₐₓ.

In an example, consider the positions of the phone illustrated in FIG. 10A. When the phone is on the sagittal plane that bisects the user's body, the ITD to the left and right ears can be considered zero. Let the corresponding azimuth angles of the phone held in the left and right hand be ϕₘ^{L} and ϕₘ^{R}. From simple geometric relationships, lₛₕ/lₛ=sin ((ϕₘ^{L}−ϕₘ^{L})/2)·cos θₘ; as illustrated in FIG. 10B. In practice, it may be difficult for a user to precisely place the sound source 132 in the sagittal plane. The transformation module 124 can approximate such locations by interpolating locations with small ITDs when the sound source 132 is moved by both hands.

To estimate α, when the sound source 132 is on a line connecting the user's ears, the absolute value of ITD is maximized. Once such a position is identified (directly or via interpolation), the transformation module 124 can estimate α as π/2−ϕₘ. The first term is due to the fact that the azimuth angle in the HCF at this position is π/2 as illustrated in FIG. 10C.

To estimate l_{z}/lₛ, when the absolute value of ITD is maximized, l_{z}/lₛ=sin θₘ^{ref} (as illustrated in FIG. 10C). To this end, the transformation module 124 can estimate the three unknown parameters using only azimuth and elevation angles of the sound source 132 in the GCF and ITD measurements. At any position, given ϕₘ and θₘ, the transformation module 124 can then determine πₘ′ and θₘ′ using Equation (3) and Equation (4).

After training, the decoder can be used to generate HRTFs at an arbitrary direction for any subject in the training dataset. However, the decoder generally cannot be directly utilized for generating HRTFs for a new user. To do so, the HRTF measurements (represented by phases and magnitudes in frequency domain) of the user at relatively sparse locations need to be collected. The collected data can be used to adapt the decoder model for generation of the individual HRTF. For adaptation, the decoder is updated with the new user's data. In some cases, to avoid over-fitting, the decoder can be trained with both new user data, and a random batch of data from existing subjects in a dataset. In an example implementation, the random batch of data can include 5% of data in the ITA dataset, or equivalently, 5000 data entries.

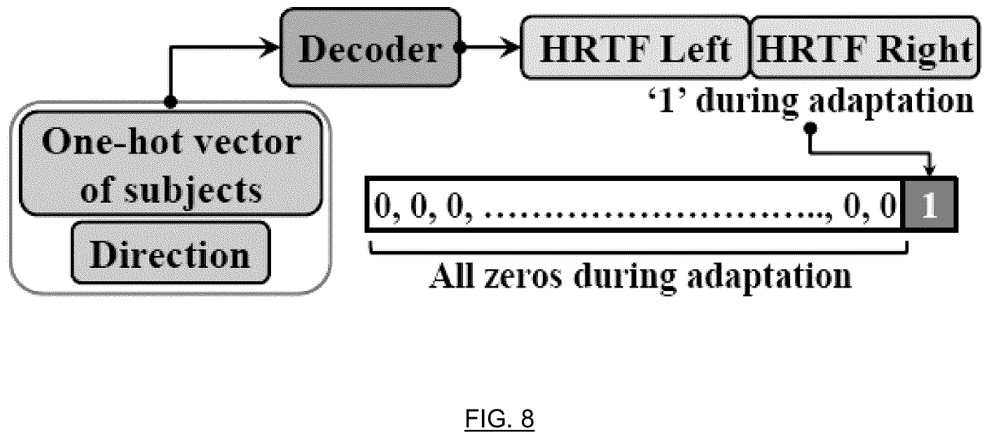

At block 308, the updating module 126 uses the positionally labeled data to adapt the decoder of the CVAE via updating to generate an individualized HRTF for the user at arbitrary directions. The updating module 126 passes a latent variable z, which is sampled from a normal Gaussian distribution, together with subject and direction vectors, as inputs to the decoder of the CVAE network to re-train the decoder. FIG. 8 illustrates a diagram of individualization of the decoder with a new user's data. As described herein, in the user vector, all elements are zero, except for the last element reserved for new users, which is set to 1. The outputs of the decoder before individualization, can be seen as a set that blends different features from all subjects in the training stage, or roughly HRTFs of an average subject.

At block 310, the output from the updated decoder is the individualized HRTF and is outputted by the output module 128 to the database 126, the network interface 110, or the user interface 106. By fine tuning the decoder parameters using data from the new user at relatively sparse directions, the locations and amplitudes of the peaks and notches in the individualized HRTF can be adapted for the new user, leveraging the structure information that the network has learned from existing training subjects.

In some cases, where the model does not itself output the time domain characteristics (as described herein), to reconstruct the time domain signals from the adapted frequency domain response through inverse Fourier transformation, phase information is generally needed. Minimum-Phase reconstruction can be used, and then an appropriate time delay (ITD) can be added to the reconstructed signals based on the direction. The ITD is estimated using the average of ITDs of all users in the dataset, and then scaled relatively to the new user base on the measurements collected (whose ITDs are known for the new user).

The present inventors performed example experiments to evaluate the performance of the present embodiments. In a first set of example experiments, the ITA dataset was used to evaluate the ability of the CVAE model to generate HRTFs for subjects. Additionally, the effects of the number of measured directions and their spatial distribution on individualizing HRTFs for new users was investigated. Out of 48 subjects in the dataset, one subject is randomly chosen for testing and data and the remaining 47 subjects are used in training the CVAE model. A small subset of the new user's data is also used for adaption and the rest is used in testing. To quantify the accuracy of the predicted HRTFs, a metric was used called Log-Spectral Distortion (LSD) defined as follows:

$$LSD ( H , \hat{H} ) = \sqrt{\frac{1}{K} {\sum }_{k = 1}^{K} {( 20 {log}_{10} \vert \frac{H ( k )}{\hat{H} ( k )} \vert )}^{2}} \qquad ( 5 )$$

where H(k) and Ĥ(k) are the ground truth and estimated HRTFs in the frequency domain, respectively, and K is the number of frequency bins. LSD is non-negative and symmetric. Clearly, if H(k) and Ĥ(k) are identical for k=1, . . . , K, LSD(H, Ĥ)=0.

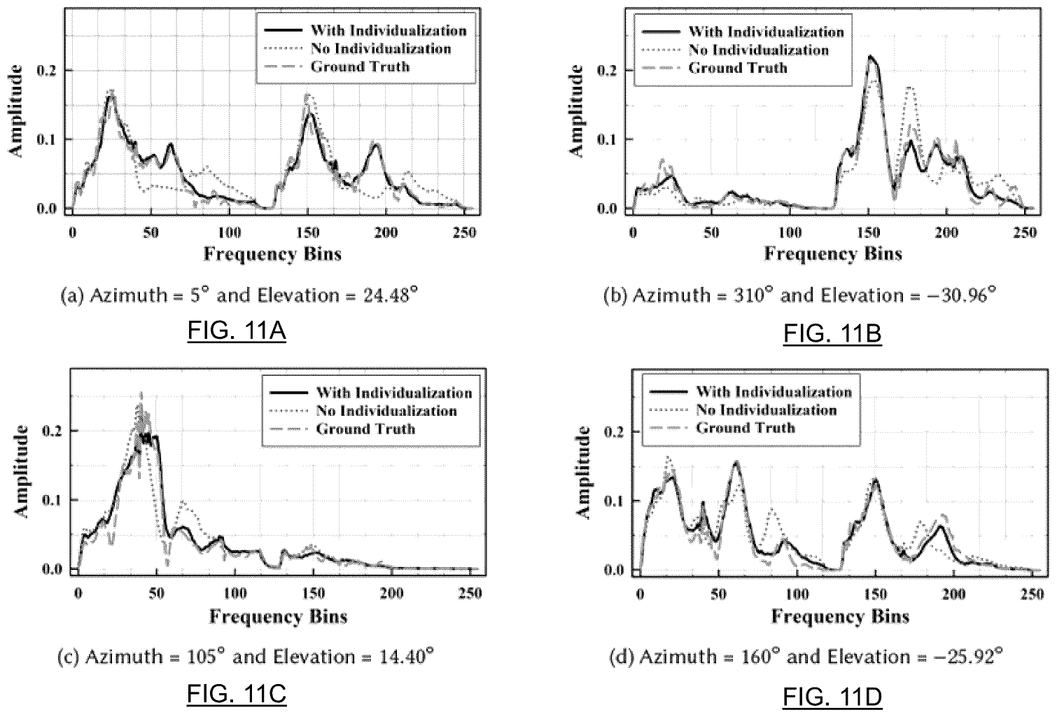

The fidelity of HRTF predictions was investigated. FIGS. 11A to 11D illustrate charts of comparisons of ground truth HRTFs and HRTFs with and without individualization for Subject 1 from the ITA dataset at four different positions/locations. Each curve concatenates the left and right HRTFs. The LSDs before individualization are: (a) 8.08, (b) 8.07, (c) 5.42, (d) 6.21, and after individualization (a) 4.62, (b) 4.25, (c) 3.47, (d) 4.14.

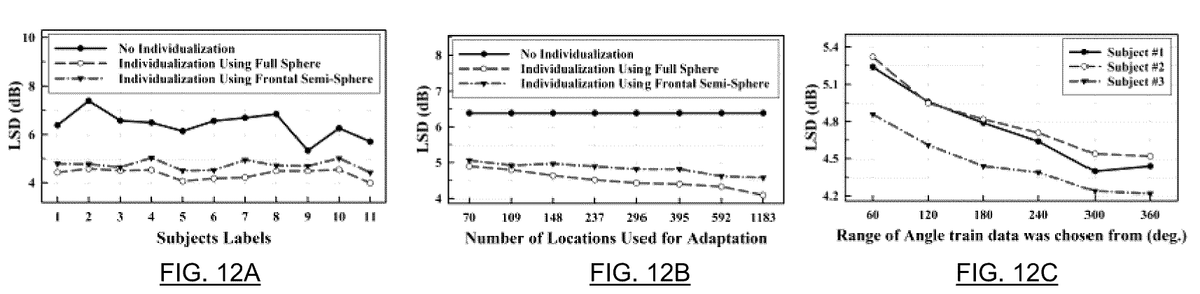

FIGS. 12A to 12C illustrate charts showing LSD errors for different subjects and with different measurement locations. In FIGS. 12A and 12B, individualization performance are shown in two cases: when the decoder is retrained using data only from the frontal semi-sphere and using data from the full sphere. FIG. 12C shows LSD errors for three subjects when the data used for individualization are chosen from a constrained azimuth angle range. The results are shown for three subjects from the ITA dataset. The error before individualization for Subjects 1 to 3 was 6.39, 7.4, and 6.15 respectively. FIG. 12A shows the LSDs for eleven subjects in the ITA dataset before and after adaptation. The lower LSDs after adaptation indicate that the proposed CVAE model and the present individualization approach can successfully generate HRTF for new users.

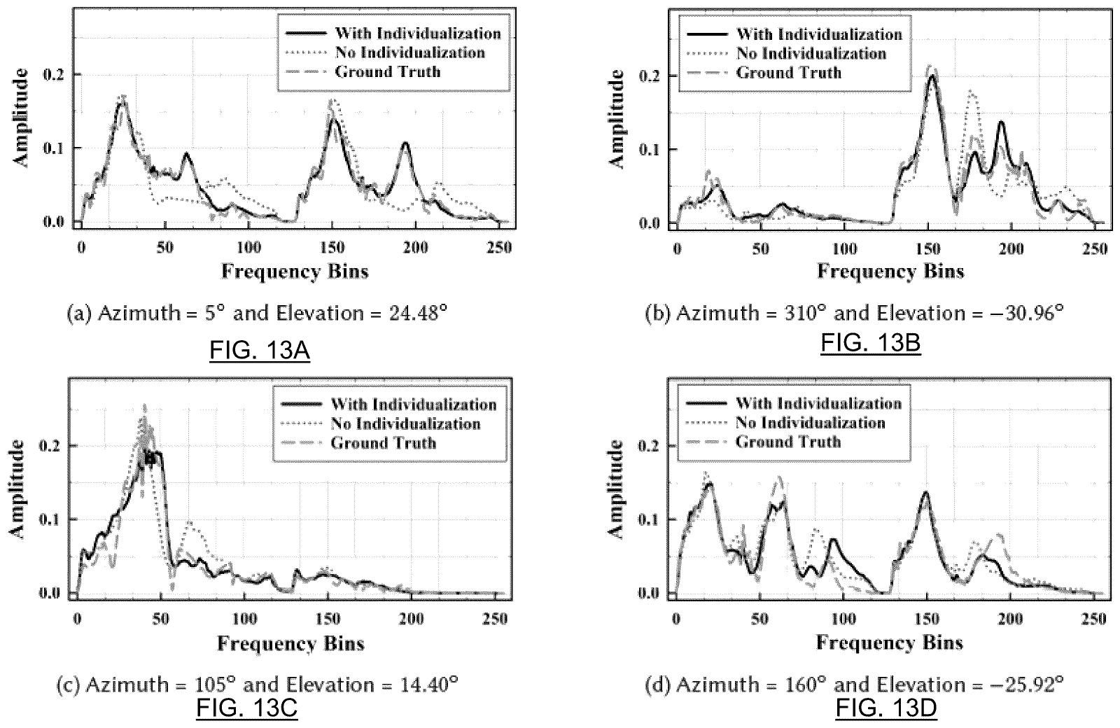

The effects of using measurements from frontal semi-spheres was investigated. As described herein, the user moves their right and left hands holding a mobile phone to obtain relatively sparse HRTF measurements. In absence of any measurement behind the user's head, it was investigated whether the present embodiments can fairly estimate HRTFs at back plane positions. To do this, the individualization step was performed, but this time using the data from the frontal semi-sphere only. FIGS. 12A to 12C compare the LSDs of individualization when data is chosen from the full sphere and when it only comes from the frontal semi-sphere. It was observed that through LSDs increase compared to the case using the full-sphere data for individualization, significant improvement can still be observed over non-individualization. FIGS. 13A to 13D show the ground truth HRTFs, and HRTFs with and without individualization. Similar to FIGS. 12A to 12C, individualization even with only data from the frontal semi-sphere can generate more accurate HRTFs than the case without individualization. FIGS. 13A to 13D show results of individualization using only HRTFs from locations in the user's frontal semisphere. Each curve concatenates HRTFs from the left and right ears. The LSD errors before individualization are: (a) 4.62, (b) 6.64, (c) 7.41, (d) 7.37, and after individualization are: (a) 4.03, (b) 4.66, (c) 6.9, (d) 6.54.

Since different people may have different range of motion of their shoulder joints, the example experiments investigated the effects of azimuth coverage on individualization. Specifically, measurements were taken only from locations whose azimuth angles fall in [−(ϕ/2, +ϕ/2], and vary p from 60° to 360°, namely, from one sixth of a full sphere to the entire sphere. The results for three subjects are shown in FIG. 12C. Clearly, as expected, when the azimuth coverage increases, LSDs drop. However, even with measurements from only one sixth of a full sphere, after individualization, LSDs are much less than those without individualization.

The effects of the sparsity of measurement locations on individualization was investigated. In this set of example experiments, the number of measurement locations were varied. As shown in FIG. 12B, fewer measurement locations degrades the performance of individualization whether they are in the frontal semi-sphere or in the full sphere. However, with as little as 70 measurement locations, 20.7% and 23.3% reductions in LSDs can be achieved in the two cases, respectively.

In the example experiments, evaluation of the accuracy of the direction finding approach and evaluation of the precision of the HRTF prediction model was investigated using real-world data.

For data capture and post-processing in the example experiments, an mobile phone application was developed, with two main functions: (1) emitting reference sounds, and (2) logging the pose of the phone in its body frame (in yaw, roll, and pitch). The sweep time of the reference exponential sweep signal was 1.2 seconds, with instantaneous frequency from 20 Hz to 22 KHz. With 1 extra second between consecutive measurements to let reverberations settle down, measuring 100 locations took about 220 seconds, a little less than 4 minutes. Two electret microphones soldered into a headphone audio jack were connected to a computer sound card for audio recording. The microphones were chosen to have good responses in human hearing ranges 20 Hz˜20 KHz. Data post-processing was performed to extract the impulse response. It is noted that the above can also be implemented in any suitable arrangement, such as on Bluetooth earphones that stream recorded audio to the phone, where the post-processing is performed on the phone.

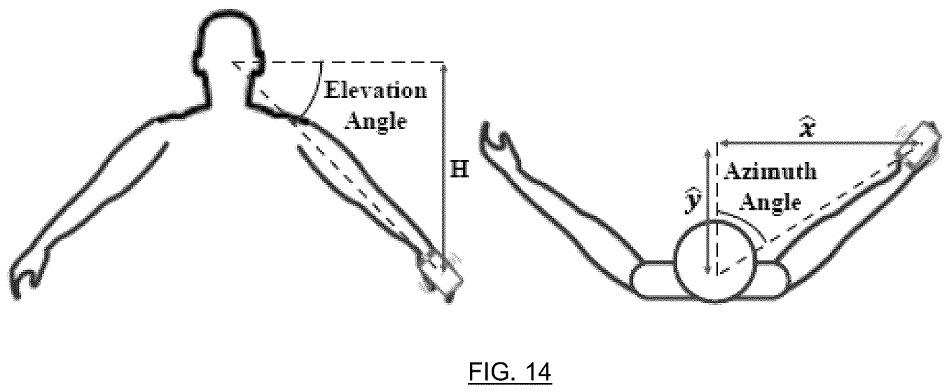

To determine ground truth for sound source directions, subjects were asked to stand on a marker on the ground, hold the mobile phone in their hand and point in different directions; as illustrated in FIG. 14. At each position, the pre-recorded sound was emitted. A measurement tape was used to determine the vertical distance (H) of the mobile phone to the centre of the user's head, and its \hat{x} and ŷ coordinates in the horizontal plane with origin at the body center and X-axis in the lateral direction and away from the body. From the measurements, the azimuth and elevation angles were calculated as:

$$Azimuth = arc tan ( \frac{\hat{x}}{\hat{y}} ) \qquad ( 6 )$$

$$Elevation = arctan ( \frac{H}{\sqrt{{\hat{x}}^{2} + {\hat{y}}^{2}}} )$$

The measurements were performed for 10 different subjects, and one manikin, which was used to eliminate human errors such as undesired shoulder or elbow movements during measurements. The users were 5 males and 5 females with ages from 29 to 70, and heights from 158 cm to 180 cm.

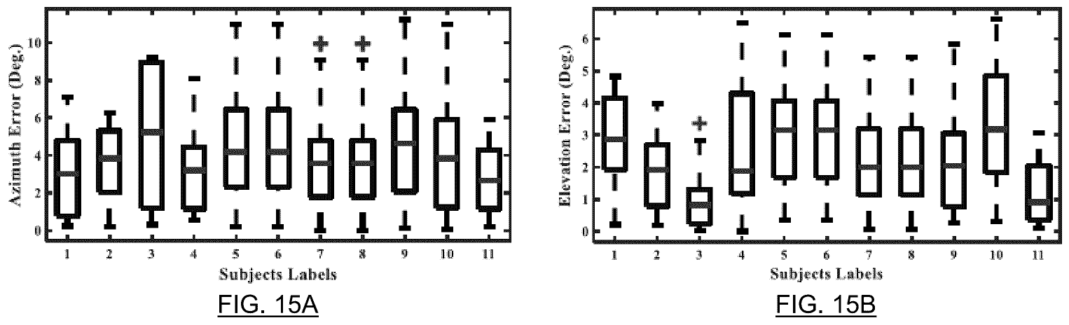

FIGS. 15A and 15B show the median, 25th, and 75th percentiles of azimuth and elevation angles estimations, respectively. In this way, FIGS. 15A and 15B show direction finding estimations for different subjects. Labels from 1 to 10 are for the human subjects, while Label 11 is for the manikin. For each box, the middle line is the median, and the bottom and top edges indicate the 25th and 75^{th} percentiles, respectively. Generally, larger errors are observed in azimuth than in elevation. This may be attributed to a larger range of motions horizontally (with both hands). By eliminating shoulder and elbow movements, the use of a manikin leads to the least angle estimation errors as expected, demonstrating the correctness of the present embodiments. More detailed results for one subject for estimations at different sound source locations are given in TABLE 1. Note even when the phone is at the same height, due to distance between the user's shoulder joint and head center, the elevation angles can differ.

TABLE 1

| Height | Azimuth | Error | Elevation | Error |
|---|---|---|---|---|
| 50.5 cm | 26.98° | −3.49° | 48.71° | −2.17° |
| | 49.81° | −5.64° | 51.64° | −1.00° |
| | 73.87° | −1.86° | 54.29° | −3.60° |
| | 100.48° | 0.18° | 57.9° | −2.14° |
| 33.0 cm | 4.04° | −5.42° | 30.64° | −0.44° |
| | 55.02° | −0.94° | 33.46° | −1.92° |
| | 80.47° | 3.63° | 36.65° | −3.19° |
| | 111.49° | 6.24° | 39.99° | −3.97° |
| 21.0 cm | 2.98° | −5.98° | 19.63° | −0.36° |
| | 51.23° | −1.89° | 20.95° | −1.83° |
| | 101.18° | −4.66° | 25.14° | −3.49° |

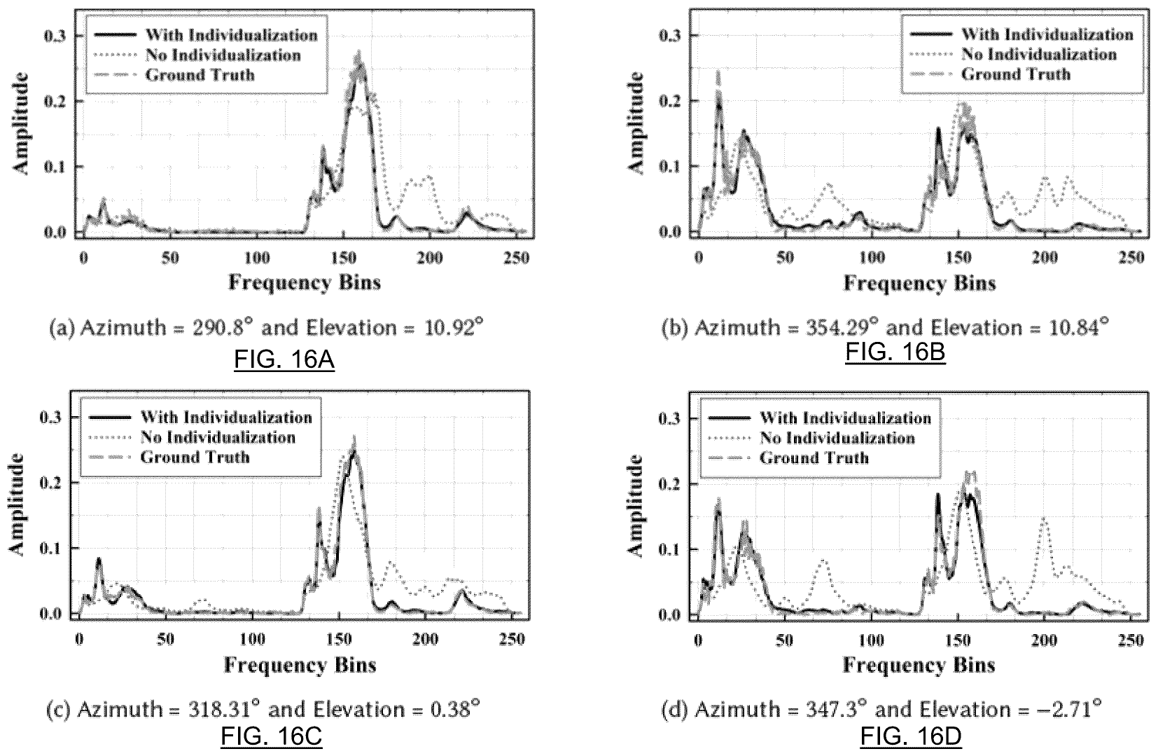

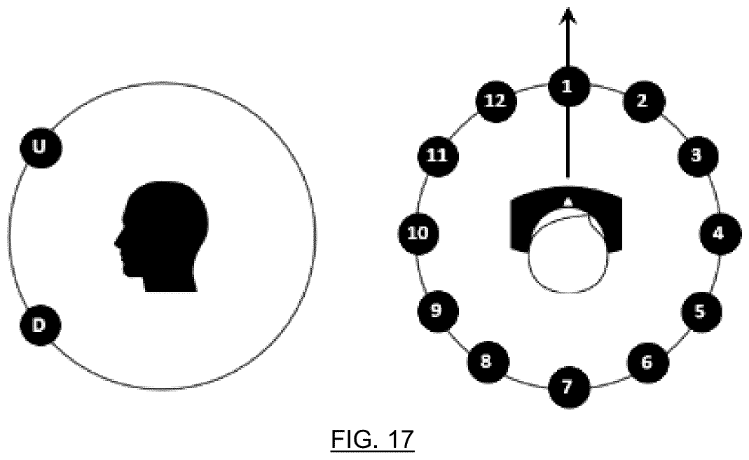

The results of individualization for one test subject are shown in FIGS. 16A to 16D. For this subject, measurements at 83 locations were collected during the experiment, 60 of which were used for individualization, and the remaining 23 locations were used for testing. Each curve concatenates HRTFs from the left and right ears. The LSD errors before individualization are: (a) 13.79, (b) 15.48, (c) 15.03, (d) 16.10, and after individualization are (a) 7.61, (b) 7, (c) 6.53, (d) 7.07. The individualized HRTFs clearly resemble the measured one more closely than without individualization in all cases. It is worth mentioning that since measurements are done in an indoor environment, the calculated HRTF is a combination of room effects, HRTFs of the test subjects, and distortions of the speaker and the microphones. Despite these challenges, the results show substantial advantages because applications of HRTFs, such as binaural localization, need to account for environment effects. Since the data acquisition for individualization in the present embodiments is fast and simple, the user can reasonably do so quickly and effectively.

The present embodiments provide substantial advantages for various applications; for example, for binaural localization and for acoustic spatialization.

For binaural localization, the example experiments randomly selected a subject and trained a localization model using the HRTF data from the user in the ITA dataset; this model is referred to as SL_{base}. A subset of the HRTF data from a different subject in the dataset, or real measurements discussed herein, were used to build a subject-specific localization model, called SL_{adapt}. The steps followed included: First, taking relatively sparse samples from the HRTF data for the new subject. Next, training an individualized HRTF decoder. The decoder is then used to generate HRTF data used to train SL_{adapt} for the new subject. For evaluation, recordings were taken of different types of sounds from the Harvard Sentences dataset and convolved with the predicted HRTFs at respective directions as test data for localization.

The model used was a fully-connected neural network, with three hidden units, with ReLU activation functions, and a dropout layer after each. The output is a classification over 36 azimuth angles represented as a one-hot vector. The network took as inputs a vector representing incoming sounds, and outputted the azimuth location. Invariant features pertaining to the location of sounds but not the types of sounds were needed. The normalized cross-correlation function (CCF) was used to compute one such feature. The CCF feature is defined as follows:

$$CCF ( τ ) = \frac{{\sum }_{m} ( {x}_{l} ( m ) - {\overline{x}}_{l} ) {\sum }_{m} ( {x}_{r} ( m - τ ) - {\overline{x}}_{r} )}{\sqrt{{\sum }_{m} {( {x}_{l} ( m ) - {\overline{x}}_{l} )}^{2}} \sqrt{{\sum }_{m} {( {x}_{r} ( m - τ ) - {\overline{x}}_{r} )}^{2}}} , \qquad ( 7 )$$

$$τ ∈ [ - {τ}_{max} , {τ}_{max} ]$$

where xₗ and xᵣ are the acoustic signals at the left and right ears, x̅ₗ and x̅ᵣ are average values of the signals over a window of size 2τₘₐₓ, m is the sample index, τ is delay in time, and τₘₐₓ is the maximum delay that a normal human can perceive, about 1 ms. For sounds sampled at 44.1 KHz, τₘₐₓ corresponds to 45 samples. Therefore, a CCF feature has a dimension of 91. The ILD feature is defined as:

$$ILD = 10 {log}_{10} \frac{{\sum }_{m} {x}_{l}^{2} ( m )}{{\sum }_{m} {x}_{r}^{2} ( m )} \qquad ( 8 )$$

with a dimension of 1. By concatenating the two, a feature vector of length 92 is the input to the neural network. Since the model can only predict azimuth angles, the location error is defined as:

$$Error = \frac{{\sum }_{n = 1}^{N} \vert θ - \hat{θ} \vert}{N} \qquad ( 9 )$$

Azimuth estimation errors are summarized in TABLE 2 for different setups. Subject A, B are both from the ITA dataset while Subject C is one of the users from whom real data was collected. In the example experiments, SL_{base} is trained on data of Subject A with three different sounds. SL_{adapt} models trained with individualized HRTF data for Subject B and Subject C, respectively. The results are averages of 1183 testing locations for each test subject.

TABLE 2

| | | Localization Error | | | |
|---|---|---|---|---|---|
| | | SL_{base}^{A} | SL_{base}^{B} | SL_{adapt}^{B} | SL_{adapt}^{C} |
| Subject A | Training Sounds | 11.22° | — | — | — |
| | Test Sound 1 | 12.17° | — | — | — |
| | Test Sound 2 | 12.95° | — | — | — |
| Subject B | Training Sounds | — | 12.1° | 13.36° | — |
| | Test Sound 1 | 18.99° | 13.19° | 14.46° | — |
| | Test Sound 2 | 19.18° | 14.02° | 13.94° | — |
| Subject C | Training Sounds | — | — | — | 9.83° |
| | Test Sound 1 | 16.42° | — | — | 11.2° |
| | Test Sound 2 | 21.38° | — | — | 13.45° |

TABLE 2 shows results before and after adaption. When Subject A's data is used for training and testing the localization model, the azimuth estimation errors are relatively low for different sounds. When the localization model trained with Subject A's HRTF data is applied to Subject B and C, the errors increase drastically. After individualization with a small amount of Subject B and C data, 5° improvement is observed for both subjects. This demonstrates the substantial effectiveness of individualized HRTFs.

Acoustic spatialization is another application that can benefit from individualized HRTFs. Acoustic spatialization customizes the playbacks of sounds in a listener's left and right ears to create 3D immersive experiences. In this example experiment, after collecting data from the users by measuring their HRTFs at relatively sparse locations, subject-dependent decoders are trained to generate their respective HRTFs in different directions.

For each subject, 14 sound files were prepared by convoluting a mono sound (e.g., a short piece of music) with individualized HRTFs at directions chosen randomly from 12 azimuth angles evenly distributed between 0° and 360°, and two elevation angles; as exemplified in the diagram of FIG. 17. Additionally, sound files were prepared by convoluting the same sound with HRTFs of an arbitrary subject in the ITA dataset at different azimuth and elevation angles. The two sets of sound files were then mixed and shuffled. The user was then asked to play back all sounds using a headset and label their perceived sound locations among the possible azimuth and elevation angles. This procedure was repeated for all subjects. It was determined that with the individualized HRTFs, subjects were accurately able to detect the correct azimuth angles 82.55% of the time on average. The accuracy drops to a mere 29.17% of the time when the unmatched HRTFs are used. While subjects reported difficulties in determining elevation angles, this is consistent with the general fact that human auditory systems generally have poor elevation resolution. Therefore, the HRTF individualization provided more accurate acoustic spatialization, and thus, a better 3D immersion experience to users.

The example experiments illustrate the substantial advantages of the present embodiments in providing an approach to HRTF individualization using only sparse data from the users. In some cases, a quick and efficient data collection procedure can be performed by users, at any setting, without specialized equipment. The present embodiments shows great improvements in adaptation time compared to perceptual-based methods. Accuracy of the present embodiments has been investigated in the example experiments using both a public dataset and real-world measurements. The advantages of individual HRTFs have been demonstrated in the example experiments using binaural localization and acoustic spatialization applications.

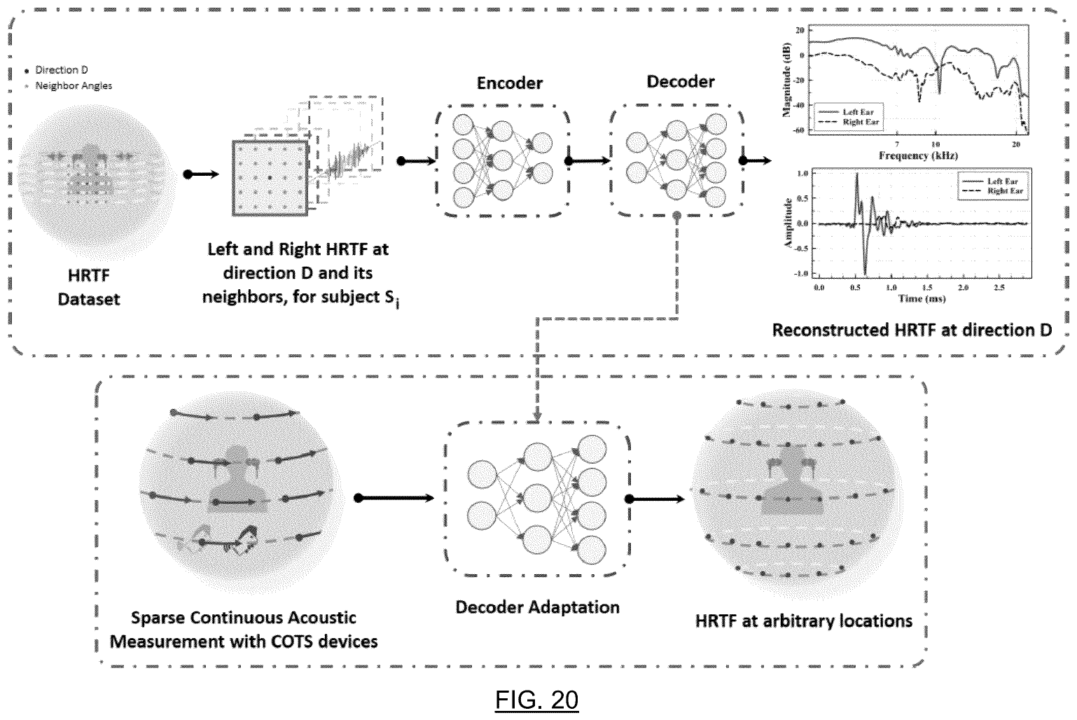

As an illustrative example, FIG. 20 illustrates a diagram of HRTF individualization, in accordance with the present disclosure. Sparse measured data (either continuously measured or measured at arbitrary locations) are used to adapt only the decoder (from the autoencoder architecture) for subjects, which can then generate HRTF of the subject at arbitrary locations.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

1. A computer-executable method for determining an individualized head related transfer functions (HRTF) for a user, the method comprising:
receiving measurement data from the user, the measurement data generated by repeatedly emitting an audible reference sound at positions in space around the user and, during each emission, recording sounds received near each ear of the user, the measurement data comprising, for each emission, the recorded sounds and positional information of the emission;
determining the individualized HRTF by updating a decoder of a trained generative artificial neural network model, the decoder receives the measurement data as input, the trained generative artificial neural network model comprising an encoder and the decoder, the generative artificial neural network model is trained using data gathered from a plurality of test subjects with known spectral representations and directions for associated HRTFs at different positions in space; and
outputting the individualized HRTF.

2. The method of claim 1, wherein the positions in space around the user comprise a plurality of fixed positions.

3. The method of claim 1, wherein the positions in space around the user comprise positions that are moving in space.

4. The method of claim 1, wherein the audible reference sound comprises an exponential chirp.

5. The method of claim 1, wherein the generative artificial neural network model comprises a conditional variational autoencoder.

6. The method of claim 5, wherein training of the conditional variational autoencoder comprises using the data gathered from the plurality of test subjects to learn a latent space representation for HRTFs at different positions in space.

7. The method of claim 6, wherein the decoder reconstructs an HRTF for the user's left ear and an HRTF for the user's right ear at a given direction from the latent space representation.

8. The method of claim 6, wherein a sparsity mask is input to the decoder to indicate a presence or an absence of parts of temporal data of the reference sound in a given direction.

9. The method of claim 1, wherein the individualized HRTF comprises magnitude and phase spectra.

10. The method of claim 9, wherein the phase spectra is determined by the generative artificial neural network model by learning real and imaginary parts of a Fourier transform of the HRTFs separately.

11. The method of claim 9, wherein an impulse response for the individualized HRTF is determined by applying an inverse Fourier transform on a combination of the magnitude and phase spectra.

12. A system for determining an individualized head related transfer functions (HRTF) for a user, the system comprising a processing unit and data storage, the data storage comprising instructions for the one or more processors to execute:
a measurement module to receive measurement data from the user, the measurement data generated by repeatedly emitting an audible reference sound by a sound source at positions in space around the user and, during each emission, recording sounds received near each ear of the user by a sound recording device, the measurement data comprising, for each emission, the recorded sounds and positional information of the sound source;
a machine learning module to determine the individualized HRTF by updating a decoder of a trained generative artificial neural network model, the decoder receives the measurement data as input, the trained generative artificial neural network model comprising an encoder and the decoder, the generative artificial neural network model is trained using data gathered from a plurality of test subjects with known spectral representations and directions for associated HRTFs at different positions in space; and
an output module to output the individualized HRTF.

13. The system of claim 12, wherein the positions in space around the user comprise a plurality of fixed positions.

14. The system of claim 12, wherein the positions in space around the user comprise positions that are moving in space.

15. The system of claim 12, wherein the sound source is a mobile phone and the sound recording device comprises in-ear microphones.

16. The system of claim 12, wherein the generative artificial neural network model comprises a conditional variational autoencoder.

17. The system of claim 16, wherein training of the conditional variational autoencoder comprises using the data gathered from the plurality of test subjects to learn a latent space representation for HRTFs at different positions in space.

18. The system of claim 17, wherein the decoder reconstructs an HRTF for the user's left ear and an HRTF for the user's right ear at a given direction from the latent space representation.

19. The system of claim 17, wherein a sparsity mask is input to the decoder to indicate a presence or an absence of parts of temporal data of the reference sound in a given direction.

20. The system of claim 12, wherein the individualized HRTF comprises magnitude and phase spectra.

21. The system of claim 20, wherein the phase spectra is determined by the generative artificial neural network model by learning real and imaginary parts of a Fourier transform of the HRTFs separately.

22. The system of claim 20, wherein an impulse response for the individualized HRTF is determined by applying an inverse Fourier transform on a combination of the magnitude and phase spectra.
